(19) United States
(12) Patent Application Publication
(10) Pub. No.: US 2003/0060764 A1
(43) Pub. Date: Mar. 27, 2003

(54) Multi-functional nasogastric tubular device for use with patients undergoing general anesthesia

(76) Inventors: Rup K. Dua, Pittsburgh, PA (US); Alexander H. Georgiades, Pittsburgh, PA (US); George J. Peters, Pittsburgh, PA (US); Michael R. Swartz, Pittsburgh, PA (US)

Correspondence Address: Michael R. Swartz, Esq., 205 Royal Oak Avenue, Pittsburgh, PA, 15235, US

(21) Appl. No.: 10/230,048

(22) Filed: Aug. 28, 2002

Related U.S. Application Data
(60) Provisional application No. 60/316,432, filed on Aug. 30, 2001.

(51) Int. Cl. A61M 1/00

(52) U.S. Cl. 604/129

(57) ABSTRACT

A multi-functional nasogastric tubular device for insertion into a patient includes an elongated flexible body having a pair of spaced apart opposite proximal and distal ends, a plurality of spaced apart lumens formed in the body so as to extend side-by-side with respect to one another between the proximal and distal ends of the body and being open at least at the proximal end of the body, and a temperature sensor disposed on the body, such as in one of the lumens of the body, and running from the proximal end of the body to the distal end thereof such that the temperature sensor is adapted to monitor internal core temperature of a patient and also to serve as a radiopaque element for marking the position of the elongated body in the patient.

This utility patent application claims the benefit of provisional application No. 60/316,432 filed Aug. 30, 2001.

### BACKGROUND OF THE INVENTION

1. Field of the Invention

The present invention generally relates to medical devices used in providing medical care to a patient undergoing general anesthesia or ICU (Intensive Care Unit) and, more particularly, is concerned with a multi-functional nasogastric tubular device for removing gastric contents from a patient's stomach, monitoring core body temperature of the patient, and providing a radiopaque marker of the position of the tubular device in the patient.

2. Description of the Prior Art

When a patient undergoes general anesthesia in conjunction with medical procedures, such as laparoscopic and open abdominal surgery, to treat various medical problems, for example surgical peptic ulcers or intestinal obstruction, the standard of care in monitoring that patient involves the placement of a nasogastric (NG) tube for the removal of gastric contents in order to prevent aspiration and the placement of a temperature sensor (TS) tube which carries a thermistor in order to monitor the core body temperature of the patient. Heretofore, each of these tubes, being made of flexible plastic material, has been inserted by a physician, or his appointee, separately through one of the two nostrils of the patient and then extended similarly through the oropharynx and esophagus to the stomach of the patient. The NG tube, generally from 36 to 48 inches in length, may extend into the stomach or further into the gastrointestinal tract. The TS tube, being typically shorter in length than the NG tube, typically terminates above the stomach.

The distal end of the NG tube, located in the stomach, includes several open suction ports or openings for permitting the passage of gastric fluids into the tube. The proximal end of the NG tube, extending from the one nostril, is typically pulled to the side of a patient's head and taped to the patient's skin and is normally connected through a collector vessel to a suction source. Thus, the gastric fluids are drawn through the openings in the distal end, through the tube, to the collector vessel. A typical NG tube is the one disclosed in U.S. Pat. No. 5,417,664 assigned to C. R. Bard, Inc. The Bard NG tube is a double lumen sump tube, having a large lumen and a smaller lumen. The large lumen is used for suction drainage and irrigation and is connected to the suction source providing either intermittent or continuous suction. The smaller lumen vents the suction drainage lumen to the atmosphere through a perforation or opening in the distal end of the NG tube.

As mentioned above, in conjunction with the use of a NG tube it is typical to also monitor the core temperature of a patient, either externally or internally. External monitoring of temperature, taken such as by skin probes, etc., is not as accurate and does not provide the true core temperature of a patient. Consequently, internal core temperature sensors are being used more commonly. A typical TS tube for taking internal core body temperature is the one sold by Mallinckrodt Inc. of St. Louis, Mo. under the trademark “Mon-a-therm”. This TS tube has its distal end, located in the esophagus, provided with small openings and its opposite proximal end, extending from the other nostril, provided with a connection stopper for connection to a stethoscope.

U.S. Pat. Nos. 5,186,172 and 5,456,251 to Fiddian-Green discloses a tonometric catheter device having a multipassage tubing which can be configured to serve as a nasogastric sump, either with or without gastric suction. The tubing has three individual noncommunicating (between each other) passageways or lumens, namely, an air lumen, an optional suction lumen and a tonometric catheter lumen. A membrane surrounds an intermediate portion of the catheter device and perforations are formed in the tubing to provide communication between the catheter lumen and a sampling chamber defined by the membrane. Also, a conductor wire can be routed through the tonometric catheter lumen to one or more sensor elements bonded to either the inner or outer wall of the tubing for detecting a property indicative of pH and/or temperature. For sensing temperature, a thermistor can be used as the temperature sensor element.

The Fiddian-Green device thus discloses a single tubing with three lumens and a conductor wire and a temperature sensor element disposed in one of the lumens. It also discloses that the device can serve as a NG device where the function of a NG tube having two lumens is combined with the function of a TS tube having one lumen and a wire and a temperature sensor element disposed therein. However, in the Fiddian-Green device the temperature sensor element is located intermediately between the opposite ends of its lumen and thus the wire leading from the proximal end of the lumen to the sensor element does not extend past the sensor element to the distal end of the lumen.

In the prior art, it is known to provide a radiopaque stripe along the outside of the NG tube, extending between the opposite ends thereof, so that an X-ray can be taken to see exactly where the tube is located in the patient's stomach. This function is also disclosed in Fiddian-Green device by use of the radiopaque stripe along the device or by fitting of a tungsten rod as an end plug in the end of the air lumen.

While the Fiddian-Green device may constitute a step in the right direction by combining separate functions performed by separate devices into a single device, it is the perception of the inventor herein that this device does not go far enough and thus a need still exists for an innovation that will provide a more comprehensive solution to the problems of the prior art.

### SUMMARY OF THE INVENTION

The present invention provides a nasogastric tubular device designed to satisfy the aforementioned need. The nasogastric tubular device of the present invention is multi-functional in that it can be used for performing multiple functions concurrently, namely, removing gastric contents from a patient's stomach, monitoring core body temperature of the patient, and providing a radiopaque marker of the position of the tubular device in the patient. In the tubular device of the present invention, the need for a separate radiopaque marker such as in the form of a radiopaque stripe or a tungsten plug is eliminated and, instead, the wires of the temperature sensor extend the entire length of the tubing, from the proximal end to the distal end thereof, so as to combine into one component the radiopaque marker function and the core body temperature monitoring function.

By combining the aforementioned three functions into the single nasogastric tubular device of the present invention, the steps a physician has to perform are reduced and a portion of the time spent by the physician engaged with such steps is thereby saved. Also, the patient will now experience less discomfort in not having separate NG and TS tubes inserted through and extending from his or her separate nostrils.

Accordingly, the present invention is directed to a multi-functional nasogastric tubular device for insertion into a human patient. The multi-functional device comprises: (a) an elongated flexible body having a pair of spaced apart opposite proximal and distal ends; (b) a plurality of spaced apart lumens formed in the body so as to extend side-by-side with respect to one another between the proximal and distal ends of the body and being open at least at the proximal end of the body; and (c) a temperature sensor disposed on the body, such as in one of the lumens of the body, and running from the proximal end of the body to the distal end thereof such that the temperature sensor is adapted to monitor internal core temperature of a patient and also to serve as a radiopaque element for marking the position of the elongated body in the patient.

More particularly, the plurality of lumens include a first lumen being adapted for use in suctioning gastric contents from a stomach of a patient and a second lumen being adapted for providing communication of air between outside of said body and the stomach of the patient. The plurality of lumens also includes a third lumen having the temperature sensor disposed therein.

Further, the temperature sensor includes a temperature sensing element disposed along the body at a location intermediately between and spaced from the proximal and distal ends of the body. The temperature sensor further includes a pair of wires extending from the proximal end of the body to the temperature sensing element and being operatively connected to the temperature sensing element so as to be capable of transmitting an electrical signal between the temperature sensing element and a location outside of the body. The temperature sensor further includes another wire separate from the pair of wires. The another wire extends at least from the temperature sensing element to the distal end of the body such that at least a portion of the wires serves as the radiopaque element extending from the proximal end to the distal end of the body. Also, the another wire can extend from the proximal end of the body to the distal end thereof. The another wire preferably is a dummy wire not operatively connected to the temperature sensing element.

These and other features and advantages of the present invention will become apparent to those skilled in the art upon a reading of the following detailed description when taken in conjunction with the drawings wherein there is shown and described an illustrative embodiment of the invention.

### BRIEF DESCRIPTION OF THE DRAWINGS

In the following detailed description, reference will be made to the attached drawings in which:

FIG. 1 is a foreshortened plan view of a multi-functional nasogastric tubular device of the present invention.

FIG. 2 is a fragmentary plan view of a lower portion of an elongated rod-shaped flexible body of the device after being rotated ninety degrees from the orientation shown in FIG. 1.

FIG. 3 is a partial diagrammatic view showing the device of FIG. 1 inserted into the stomach of the human body.

FIG. 4 is an enlarged fragmentary front elevational view of a proximal end of the body of the device as seen along line 4-4 of FIG. 1 showing a main suction lumen, a smaller vent lumen and a still smaller temperature sensor lumen formed in and extending through the body of the device between proximal and distal ends of the device, also showing a pair of electrical signal transmitting wires of a temperature sensor disposed in the temperature sensor lumen of the flexible body.

FIG. 5 is an enlarged fragmentary plan view of the body of the device showing the portion thereof encompassed by a curved arrow 5 in FIG. 1 which has a side opening in communication with the temperature sensor lumen of the body and an outer wrap extending thereabout and overlying the side opening.

FIG. 6 is a longitudinal sectional view of the portion of the body of the device shown in FIG. 5, now showing the thermistor of the temperature sensor disposed adjacent to the side opening.

FIG. 7 is an enlarged fragmentary plan view of the portion of the body of the device shown in FIG. 5 but with the outer wrap of FIG. 5 being omitted.

FIG. 8 is an enlarged cross-sectional view of the body of the device taken along line 8-8 of FIG. 6 upstream from the thermistor, showing the electrical signal transmitting wires which run through the temperature sensor lumen to the thermistor from the proximal end of the body of the device as shown in FIG. 1 and are operatively connected to the thermistor.

FIG. 9 is an enlarged cross-sectional view of the body of the device taken along line 9-9 of FIG. 6 through the thermistor.

FIG. 10 is an enlarged cross-sectional view of the body of the device taken along line 10-10 of FIG. 6 downstream from the thermistor, showing a single non-transmitting or dummy wire which runs through the temperature sensor lumen from the thermistor to the distal end of the body of the device and is inoperatively connected to the thermistor.

FIG. 11 is an enlarged cross-sectional view of the body of the device taken along line 11-11 of FIG. 2 at the distal end thereof where the respective lumens therein terminate and the body thus has a solid form.

FIG. 12 is an enlarged cross-sectional view of the body of the device taken along line 12-12 of FIG. 2, showing the dummy wire disposed in the temperature sensor lumen of the body and a cross hole formed in the body so as to provide communication of the suction and vent lumens with one another and with the outside of the body.

FIG. 13 is an enlarged cross-sectional view of the body of the device taken along line 13-13 of FIG. 1, showing one of first and second pluralities of transfer holes formed in a staggered offset relationship from one another on opposite sides of the body so as to provide communication of the outside of the body only with the suction lumen therein.

FIG. 14 is an enlarged cross-sectional view of the body of the device taken along line 14-14 of FIG. 1, showing another of the transfer holes which is on an opposite side of the body from the side of the one transfer hole shown in FIG. 13.

FIG. 15 is an enlarged cross-sectional view of the body of the device taken along line 15-15 of FIG. 1, showing still another of the transfer holes which is on the same side of the body as the one transfer hole shown in FIG. 13.

FIG. 16 is a fragmentary longitudinal sectional view of the body of the device taken along line 16-16 of FIG. 17 showing the pair of electrical signal transmitting wires of the temperature sensor disposed in the temperature sensor lumen of the body.

FIG. 17 is an enlarged cross-sectional view of an upper portion of the body of the device taken along line 17-17 of FIG. 1 showing an oval configuration of the upper portion of the body disposed adjacent to the proximal end of the device.

FIG. 18 is a foreshortened plan view of the temperature sensor used in the multi-functional device of the present invention.

FIG. 19 is an enlarged, fragmentary plan view of the body of the device showing the layout of the plurality of transfer holes with drainage grooves or channels formed in the opposite sides of the body between and interconnecting the transfer holes along the opposite sides of the body.

FIG. 20 is a cross-sectional view of the body of the device taken along line 20-20 of FIG. 19, showing a generally V-shaped cross-sectional configuration of one of the drainage channels.

FIG. 21 is a fragmentary longitudinal sectional view of the body of the device similar to that of FIG. 6, now showing an alternative form of the temperature sensor disposed in the temperature sensor lumen of the body wherein the dummy wire is disposed with the electrical signal transmitting wires running from the proximal end of the device to the thermistor but is not connected to the thermistor and instead runs past the thermistor toward the distal end of the device.

### DETAILED DESCRIPTION OF THE INVENTION

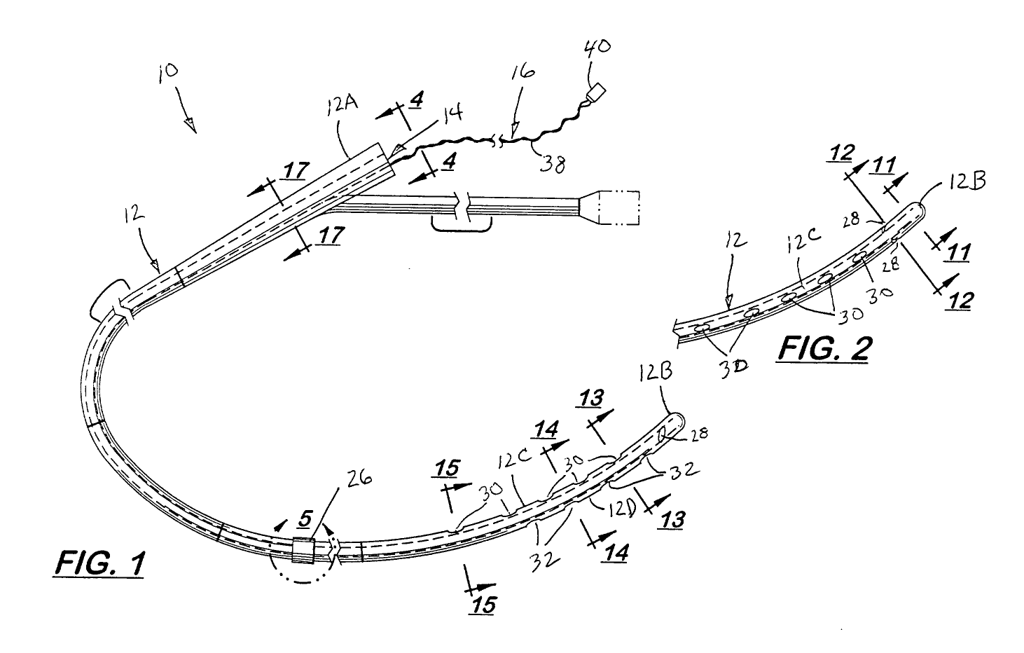

Referring to the drawings and particularly to FIGS. 1 and 2, there is illustrated a multi-functional nasogastric tubular device of the present invention, generally designated 10. As best seen in FIG. 3, the multi-functional nasogastric tubular device 10 is adapted for insertion through a single nostril N, the oropharynx O and the esophagus E, and into the stomach S of a human patient P.

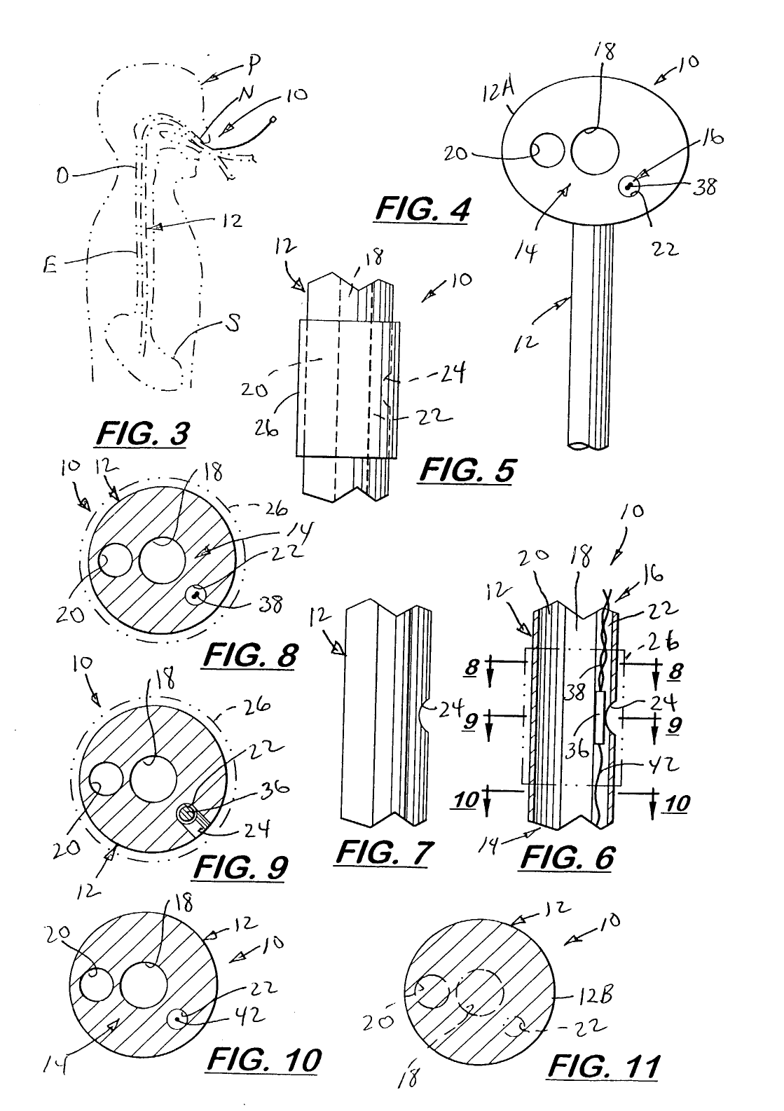

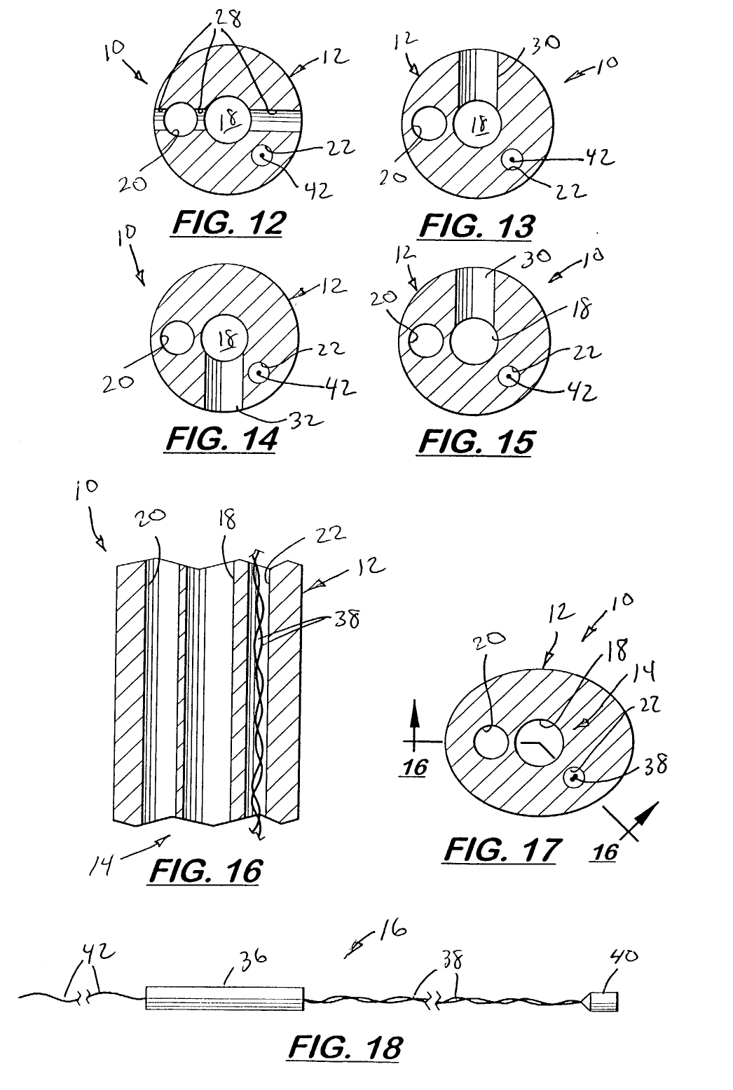

Referring to FIGS. 1, 2 and 4-17, the multi-functional device 10 basically includes an elongated flexible body 12 having a pair of spaced apart opposite proximal and distal ends 12A, 12B, a plurality of passageways or lumens 14 formed internally and laterally spaced apart to one another in the body 12 so as to co-extend in a side-by-side relationship with respect to one another between the proximal and distal ends 12A, 12B of the body 12, with the lumens 14 being open at least at the proximal end 12A of the body 12, and means in the form of a temperature sensor 16 disposed on the body 12, such as in one of the lumens 14 of the body 12, and running from the proximal end 12A of the body 12 to the distal end 12B thereof for monitoring internal core temperature of a patient and also for serving as a radiopaque element for marking the position of the body 12 in the patient P.

The elongated body 12 of the device 10 has an overall rod-shaped configuration and is made from a suitable plastic material, such as PVC, by using conventional fabricating techniques, such as plastic molding or extrusion. The body 12 can have any suitable cross-sectional shape, such as circular or oval, as desired. The body 12 is shown as an integral unit; however, it could be formed of three separate tubes attached together. As best seen in FIGS. 2 and 11, the termination of the distal end 12B of the body 12 is solid and thus closed such that the plurality of lumens 14 terminate at the distal end 12B of the body 12.

The plurality of internal lumens 14 provided or formed in the elongated body 12 of the device 10 preferably include a first or suction lumen 18, a second or vent lumen 20, and a third or temperature sensor lumen 22. As mentioned above, these lumens 18, 20, 22 are formed internally and in laterally spaced apart relation to one another in the body 12 so as to co-extend in the side-by-side relationship with respect to one another between the proximal and distal ends 12A, 12B of the body 12. In the illustrated embodiment, the second lumen 20 is smaller in cross-sectional size than the first lumen 18, and the third lumen 22 is smaller in cross-sectional size than the second lumen 20. The first lumen 18 is adapted for use in suctioning gastric contents from the stomach S of the patient P. The second lumen 20 is adapted for providing communication of air between outside of the elongated body 12 and the stomach S of the patient P. The third lumen 22 has the temperature sensor 16 disposed therein.

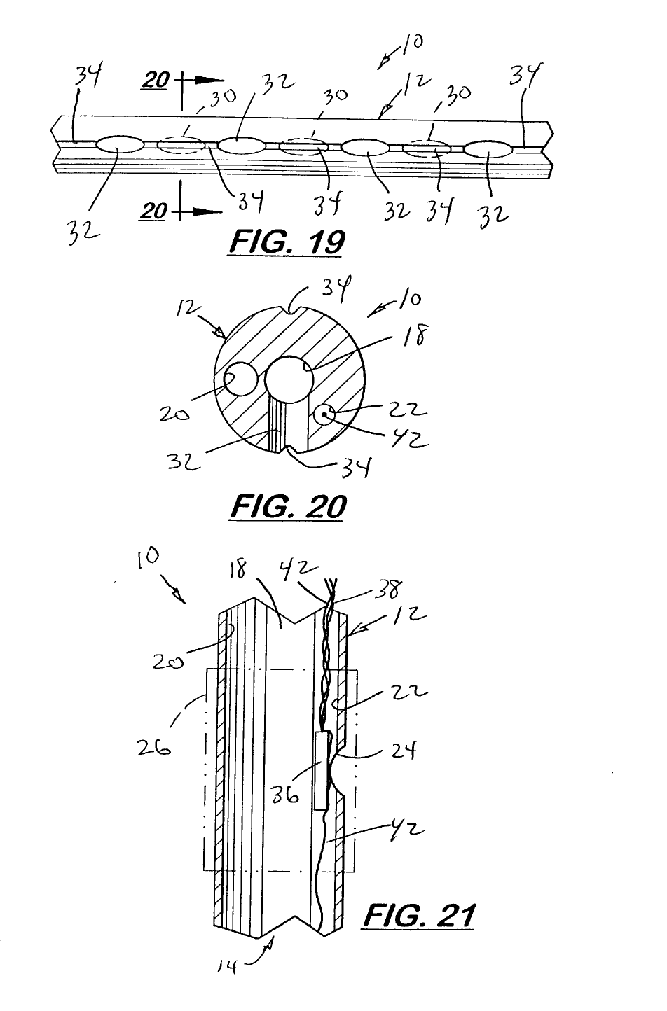

As best seen in FIGS. 5-7 and 9, the elongated body 12 of the device 10 has a side opening 24 formed therein in communication with the third lumen 22 and a protective outer wrap 26 extending about the body 12 and overlying the side opening 24. Also, as best seen in FIGS. 1, 2 and 12, near its distal end 12B the elongated body 12 has a transverse or cross hole 28 formed therein so as to provide communication of the first and second lumens 18, 20 with one another and with the stomach S of the patient P which surrounds the outside of the elongated body 12. Further, as best seen in FIGS. 1, 2 and 13-15, located upstream of the cross hole 28 the elongated body 12 has first and second pluralities of transfer holes 30, 32 formed therein, in an offset or staggered relationship with respect to one another, along opposite sides 12C, 12D of a lower portion of the body 12 so as to provide communication from outside of the body 12, and thus from the stomach S surrounding the body 12, to the first lumen 18 therein. The cross hole 28 and transfer holes 30, 32 facilitate movement of gastric contents from the stomach S into the first lumen 18 in response to creation of a suction in the first lumen 18 by well-known conventional equipment (not shown) attached to the first lumen 18 at the proximal end 12A of the elongated body 12 of the device 10. Still further, as best seen in FIGS. 19 and 20, the elongated body 12 has a plurality of drainage channels 34 formed into the opposite sides 12C, 12D of the body 12 such that each of the channels 34 extends between and interconnects adjacent pairs of the transfer holes 30, 32 of the first and second pluralities thereof along the opposite sides 12C, 12D of the body 12. Each of the channels 34 has a V-shaped cross-sectional shape although the channels 34 can be provided with other cross-sectional shapes. The channels 34 are for facilitating drainage of the gastric contents of the stomach S along the surface of the elongated body 12 to the transfer holes 30, 32.

Referring to FIGS. 1, 4, 6, 8-10 and 12-18, the temperature sensor 16 of the device 10 includes a temperature sensing element 36, such as a thermistor which is a device that is conventional and well-known per se, disposed along the body 12 at a location intermediately between and spaced from the proximal and distal ends 12A, 12B of the body 12 and preferably disposed adjacent to the side opening 24 in the body 12. By way of example, the side opening 24 and the temperature sensing element 36 are located approximately two-thirds the length of the body 12 measured from the proximal end 12A thereof such that the temperature sensing element 36 will be positioned adjacent to the upper portion of the stomach which is the optimum location for monitoring the internal core temperature of the patient P.

As best seen in FIGS. 1, 6, 8 and 16-18, the temperature sensor 16 further includes a pair of wires 38 extending from the proximal end 12A of the body 12 to the temperature sensing element 36 and being operatively connected to the temperature sensing element 36 SO as to be capable of transmitting an electrical signal between the temperature sensing element 36 and suitable well-known conventional equipment (not shown) disposed at a location outside of the body 12 and connected to an end plug 40 which is connected to the wires 38.

The temperature sensor 16 still further includes another or third wire 42 separate from the pair of wires 38 and preferably being a dummy wire which is not operatively connected to the temperature sensing element 36. As seen in a preferred embodiment of the temperature sensor 16 shown in FIGS. 6, 10, 12-15 and 18, the third wire 42 is attached to and extends from the temperature sensing element 36 to the distal end 12B of the body 12 such that at least a portion, and preferably all, of the wires serves as the radiopaque element extending from the proximal end 12A to the distal end 12B of the body 12. As seen in an alternative embodiment of the temperature sensor 16 shown in FIG. 21, the third wire 42 can also be provided to extend from the proximal end 12A of the body 12 to the distal end 12B thereof. The wires 38 of the sensor 16 and the third dummy wire 42 can be made of any suitable material, such as any conventional metallic material capable of conducting an electrical current, such as copper.

Although the nasogastric tubular device 10 of the subject invention has been described and is directed primarily for use on humans, it can also be used on animals in like fashion.

It is thought that the present invention and its advantages will be understood from the foregoing description and it will be apparent that various changes may be made thereto without departing from the spirit and scope of the invention or sacrificing all of its material advantages, the form hereinbefore described being merely preferred or exemplary embodiment thereof.

I (we) claim:

1. A multi-functional nasogastric tubular device for insertion into a patient, said device comprising:
(a) an elongated flexible body having a pair of spaced apart opposite proximal and distal ends;
(b) a plurality of spaced apart lumens formed in said body so as to extend side-by-side with respect to one another between said proximal and distal ends of said body and being open at least at said proximal end of said body, a first of said lumens being adapted for use in suctioning gastric contents from a stomach of a patient and a second of said lumens being adapted for providing communication of air between outside of said body and the stomach of the patient; and
(c) a temperature sensor disposed on said body and running from said proximal end of said body to said distal end of said body such that said temperature sensor is adapted to monitor internal core temperature of a patient and also to serve as a radiopaque element for marking the position of said elongated body in the patient.

2. The device as recited in claim 1, wherein said temperature sensor includes a temperature sensing element disposed along said body at a location intermediately between and spaced from said proximal and distal ends of said body.

3. The device as recited in claim 2, wherein said temperature sensor further includes a pair of wires extending from said proximal end of said body to said temperature sensing element and being operatively connected to said temperature sensing element so as to be capable of transmitting an electrical signal between said temperature sensing element and a location outside of said body.

4. The device as recited in claim 3, wherein said temperature sensor further includes another wire separate from said pair of wires, said another wire extending at least from said temperature sensing element to said distal end of said body such that at least a portion of said wires serves as said radiopaque element extending from said proximal end to said distal end of said body.

5. The device as recited in claim 4, wherein said another wire extends from said proximal end of said body to said distal end of said body.

6. The device as recited in claim 4, wherein said another wire is a dummy wire not operatively connected to said temperature sensing element.

7. A multi-functional nasogastric tubular device for insertion into a patient, said device comprising:
(a) an elongated flexible body having a pair of spaced apart opposite proximal and distal ends;
(b) a plurality of spaced apart lumens formed in said body so as to extend side-by-side with respect one another between said proximal and distal ends of said body and being open at least at said proximal end of said body, a first of said lumens being adapted for use in suctioning gastric contents from a stomach of a patient and a second of said lumens being adapted for providing communication of air between outside of said body and the stomach of the patient; and
(c) a temperature sensor disposed in said body and extending into said body at said proximal end thereof and running to said distal end of said body such that said temperature sensor is adapted to monitor internal core temperature of a patient and also to serve as a radiopaque element for marking the position of said elongated body in the patient.

8. The device as recited in claim 7, wherein said temperature sensor includes a temperature sensing element disposed in said body at a location intermediately between and spaced from said proximal and distal ends of said body.

9. The device as recited in claim 8, wherein said temperature sensor further includes a pair of wires extending from said proximal end of said body to said temperature sensing element and being operatively connected to said temperature sensing element so as to be capable of transmitting an electrical signal between said temperature sensing element and a location outside of said body.

10. The device as recited in claim 9, wherein said temperature sensor further includes another wire separate from said pair of wires, said another wire extending at least from said temperature sensing element to said distal end of said body such that at least a portion of said wires serves as the radiopaque element extending from said proximal end to said distal end of said body.

11. The device as recited in claim 10, wherein said another wire extends from said proximal end of said body to said distal end of said body.

12. The device as recited in claim 10, wherein said another wire is a dummy wire not operatively connected to said temperature sensing element.

13. A multi-functional nasogastric tubular device for insertion into a patient, said device comprising:
(a) an elongated flexible body having a pair of spaced apart opposite proximal and distal ends;
(b) a plurality of lumens formed in said body so as to extend side-by-side with respect to one another between said proximal and distal ends of said body and separately open at said proximal end of said body, a first of said lumens being adapted for use in suctioning gastric contents from a stomach of a patient and a second of said lumens being adapted for providing communication of air between outside of said body and the stomach of the patient; and
(c) a temperature sensor disposed in one of said lumens and extending through said proximal end of said body and running within said one lumen from said proximal end to said distal end of said body such that said temperature sensor is adapted to monitor internal core temperature of a patient and also to serve as a radiopaque element for marking the position of said elongated body in the patient.

14. The device as recited in claim 13, wherein said temperature sensor includes a temperature sensing element disposed in said one lumen of said body at a location intermediately between and spaced from said proximal and distal ends of said body.

15. The device as recited in claim 14, wherein said temperature sensor further includes a pair of wires extending within said one lumen of said body from said proximal end of said body to said temperature sensing element and being operatively connected to said temperature sensing element so as to be capable of transmitting an electrical signal between said temperature sensing element and a location outside of said body.

16. The device as recited in claim 15, wherein said temperature sensor further includes another wire separate from said pair of wires, said another wire extending within said one lumen of said body at least from said temperature sensing element to said distal end of said body such that at least a portion of said wires serves as said radiopaque element extending from said proximal end to said distal end of said body.

17. The device as recited in claim 16, wherein said another wire extends from said proximal end of said body to said distal end of said body.

18. The device as recited in claim 16, wherein said another wire is a dummy wire not operatively connected to said temperature sensing element.

19. A multi-functional nasogastric tubular device for insertion into a patient, said device comprising:
(a) an elongated flexible body having a pair of spaced apart opposite proximal and distal ends;
(b) a plurality of spaced apart lumens formed in said body so as to extend side-by-side with respect one another between said proximal and distal ends of said body and being open at least at said proximal end of said body, a first of said lumens being adapted for use in suctioning gastric contents from a stomach of a patient, a second of said lumens being adapted for providing communication of air between outside of said body and the stomach of the patient, a third of said lumens being adapted for accommodating a temperature sensor; and
(c) a temperature sensor disposed in said third lumen of said body and extending into said body at said proximal end thereof and running within said third lumen from said proximal end to said distal end of said body such that said temperature sensor is adapted to monitor internal core temperature of a patient and also to serve as a radiopaque element for marking the position of said elongated body in the patient.

20. The device as recited in claim 19, wherein said temperature sensor includes a temperature sensing element disposed in said third lumen of said body at a location intermediately between and spaced from said proximal and distal ends of said body.

21. The device as recited in claim 20, wherein said temperature sensor further includes a pair of wires extending within said third lumen of said body from said proximal end of said body to said temperature sensing element and being operatively connected to said temperature sensing element so as to be capable of transmitting an electrical signal between said temperature sensing element and a location outside of said body.

22. The device as recited in claim 21, wherein said temperature sensor further includes another wire separate from said pair of wires, said another wire extending within said third lumen of said body at least from said temperature sensing element to said distal end of said body such that at least a portion of said wires serves as said radiopaque element extending from said proximal end to said distal end of said body.

23. The device as recited in claim 22, wherein said another wire extends from said proximal end of said body to said distal end of said body.

24. The device as recited in claim 22, wherein said another wire is a dummy wire not operatively connected to said temperature sensing element.

25. A multi-functional nasogastric tubular device for insertion into a patient, said device comprising:
(a) an elongated flexible body having a rod-shaped configuration and a pair of opposite proximal and distal ends;
(b) a plurality of lumens formed in a spaced apart relationship in said body so as to co-extend side-by-side with respect to one another between said proximal and distal ends of said body and separately open at said proximal end of said body; and
(c) a temperature sensor disposed in one of said lumens and extending through said proximal end of said body and running to said distal end of said body, said temperature sensor including
(i) a temperature sensing element disposed in said one lumen at a location intermediately between and spaced from said proximal and distal ends of said body,
(ii) a pair of wires disposed in said one lumen and extending through said proximal end of said body to said temperature sensing element and being operatively connected to said temperature sensing element so as to be capable of transmitting an electrical signal through said one lumen between said temperature sensing element and a location outside of said body, and
(iii) another wire separate from said pair of wires, said another wire being disposed in said lumen and extending at least from said temperature sensing element to said distal end of said body such that at least said another wire functions as a radiopaque element for marking the position of said elongated body in a patient.

26. The device as recited in claim 25, wherein said another wire extends from said proximal end of said body to said distal end of said body.

27. The device as recited in claim 25, wherein said another wire is a dummy wire not operatively connected to said temperature sensing element.

28. The device as recited in claim 25, wherein said body has a side opening formed therein in communication with said one lumen of said body and an outer wrap extending about said body and overlying said side opening.

29. The device as recited in claim 28, wherein said temperature sensing element is a thermistor being disposed adjacent to said side opening.

30. The device as recited in claim 25, wherein said plurality of lumens include a first lumen being adapted for use in suctioning gastric contents from a stomach of a patient and a second lumen being adapted for providing communication of air between outside of said body and the stomach of the patient.

31. The device as recited in claim 30, wherein said body has a cross hole formed therein so as to provide communication of said first and second lumens with one another and with outside of said body.

32. The device as recited in claim 30, wherein said second lumen is smaller in cross-sectional size than said first lumen.

33. The device as recited in claim 30, wherein said temperature sensor is disposed in said first lumen.

34. The device as recited in claim 30, wherein said plurality of lumens also includes a third lumen being said one lumen having said temperature sensor disposed therein.

35. The device as recited in claim 34, wherein said third lumen is smaller in cross-sectional size than said second lumen.

36. The device as recited in claim 35, wherein said second lumen is smaller in cross-sectional size than said first lumen.

37. The device as recited in claim 34, wherein said body has a side opening formed therein in communication with said third lumen of said body and an outer wrap extending about said body and overlying said side opening.

38. The device as recited in claim 37, wherein said temperature sensing element is a thermistor being disposed adjacent to said side opening.

39. The device as recited in claim 30, wherein said body has a plurality of transfer holes formed in said body along at least one side of said body so as to provide communication from outside of said body to said first lumen therein.

40. The device as recited in claim 39, wherein said body further has a plurality of drainage channels formed in said one side of said body such that each of said channels extends between and interconnects adjacent pairs of said transfer holes along said one side of said body.

41. The device as recited in claim 40, wherein each of said channels has a V-shaped cross-sectional shape.

42. The device as recited in claim 30, wherein said body has first and second pluralities of transfer holes formed in said body in an offset staggered relationship with respect to one another along opposite sides of said body so as to provide communication from outside of said body to said first lumen therein.

43. The device as recited in claim 42, wherein said body further has a plurality of drainage channels formed in said opposite sides of the body such that each of said channels extends between and interconnects adjacent pairs of said transfer holes of said first and second pluralities thereof along said opposite sides of said body.

44. The device as recited in claim 43, wherein each of said channels has a V-shaped cross-sectional shape.
